(19) United States
(12) Patent Application Publication
(10) Pub. No.: US 2011/0092442 A1
(43) Pub. Date: Apr. 21, 2011

(54) Anticancer agent containing bl-angiostatin

(75) Inventors: Keiji Hasumi, Tokyo (JP); Kosuke Shimizu, Tokyo (JP); Tetsuro Yamamoto, Tokyo (JP)

(73) Assignee: TTC CO., LTD, SHIBUYA-KU TOKYO (JP)

(21) Appl. No.: 10/590,342

(22) PCT Filed: Feb. 24, 2004
(86) PCT No.: PCT/JP04/02124

§ 371 Date: Aug. 24, 2006

(51) Int. Cl. A61K 38/17; C07K 1/04; A61P 35/00

(52) U.S. Cl. 514/19.4; 514/19.3; 530/394

(57) ABSTRACT

An anticancer agent containing BL-angiostatin as an active ingredient; and a process for producing the same. Also a method for treating cancer comprising administering to a mammal a pharmaceutically effective amount of BL-angiostatin.

### TECHNICAL FIELD

The present invention relates to an anticancer agent containing BL-angiostatin, and a production method thereof.

### BACKGROUND ART

Since substances that impair vascular neogenesis inhibit the proliferation, invasion and metastasis of cancer, they are expected to be used as antitumor agents, and angiostatin is known to be such a substance. Angiostatin is a protein having a molecular weight of about 40,000 daltons which is present in the blood, and is obtained by decomposition of the fibrolytic factor, plasminogen. Angiostatin has been reported to demonstrate dramatic effects against cancer in animal studies (M. S. O'Reilly et al., Cell, 79, 315-328 (1994)).

There are known to be two types of angiostatin consisting of (1) that formed by the hydrolysis of plasminogen by elastase (M. S. O'Reilly et al., Nature Medicine, 2, 689-692 (1996)), and (2) that produced directly in yeast using genetic recombination technology (B. K. Sim et al., Cancer Research 57, 1329-1334 (1997)).

In the case of (1), however, since a large number of by-products are formed due to the low substrate specificity of elastase, thereby making it difficult to selectively form angiostatin from plasminogen, this type has the shortcoming of the activity of the resulting angiostatin being low. In addition, in the case of (2), purification of the angiostatin is both difficult and expensive, while also having the problem of low solubility in water.

Recently, an angiostatin-like fragment (mainly Glu¹-Ser⁴⁴¹, thereinafter to be referred to as “BL-angiostatin”) has been developed in which human plasminogen is specifically cleaved using bacillolysin MA, a protease produced by Bacillus megaterium strain A9542 (Japanese Unexamined Patent Publication No. 2002-272453). The amino acid sequence of human plasminogen is known, and is registered in SWISSPROT under accession number P00747. Furthermore, since the amino acid sequence of human plasminogen contains a signal peptide (Met¹-Gly¹⁹), the amino acid locations are indicated in the present application using amino acid numbers based on the mature polypeptide from which the signal peptide has been omitted, namely the amino acid sequence of Glu¹-Asn⁷⁹¹.

In addition, recombinant angiostatin exhibits greater variations in physical properties and activity than the natural type, and these are thought to be caused by such factors as changes in the three-dimensional structure based on differences in protein folding during the course of expression in different species, and differences in sugar chain modification (Cao, Y., Int. J. Biochem. Cell. Biol. 33, 357-369 (2001); Dell'Eva, R., et al., Endothelium 9, 3-10 (2002)). In addition, the solubility of recombinant proteins has also been indicated as being low for similar reasons. In consideration of these factors, the natural form produced by enzyme methods is considered to be advantageous as compared with the recombinant form.

However, since the substrate recognition of proteases is specific, it is extremely difficult to selectively produce angiostatin from plasminogen, and has the shortcoming of resulting in the formation of numerous by-products.

In addition, not all commercially available elastase standards are able to produce active angiostatin, the possibility has been suggested that angiostatin is denatured by cleavage and subsequent treatment (O'Reilly et al., Nat. Med. 2, 689-692 (1996)).

Bacillolysin MA cleaves plasminogen extremely selectively, and is able to produce BL-angiostatin (fragment having the amino acid sequence of Glu¹-Ser⁴⁴¹ as the primary product, and fragments consisting of Phe⁷⁵-Ser⁴⁴¹, Glu¹-Val⁴⁴⁹ and Phe⁷⁵-Val⁴⁹⁹ as trace products (Japanese Unexamined Patent Publication No. 2002-272453).

Moreover, the inventors of the present invention developed a bacillolysin MA-immobilized reactor (affinity trap reactor), and developed a technology for producing a plasminogen fragment having for its main component a fragment having the amino acid sequence of Glu¹-Ser⁴⁴¹ in a single step from human plasma (PCT/JP03/00338), achieving reductions in production time and costs.

In contrast to recombinant angiostatin being an internal fragment of plasminogen composed of the amino acids of Leu⁷⁴-Leu⁴⁵¹ (Sim, B. K. et al., Cancer Res. 57, 1329-1334 (1997)), BL-angiostatin, although having a different structure than the aforementioned angiostatin, demonstrates an anti-vascular endothelial cell action similar to that of angiostatin.

In particular, the primary product in the form of a fragment having an amino acid sequence of Glu¹-Ser⁴⁴¹, as well as a fragment having the amino acid sequence of Glu¹-Val⁴⁴⁹, contain the N-terminal side of plasminogen.

The N-terminal peptide of plasminogen (Glu¹-Glu⁸³) is rich in hydrophilic amino acids, and differences in the presence or absence of the N-terminal peptide are thought to result in not only differences in solubility and other physical properties, but also differences in activity and kinetics in the body, between BL-angiostatin and angiostatin.

However, there are no reports describing that BL-angiostatin actually has anticancer effects in vivo, and the development of an anticancer agent which is inexpensive, exhibits high solubility in water and has superior anticancer effects, and the development of a production method thereof, are desired.

As a result of conducting extensive research on the effects of the aforementioned BL-angiostatin on cancer, the inventors of the present invention surprisingly found that, since BL-angiostatin has extremely superior anticancer effects and exhibits high solubility in water, it demonstrates extraordinary anticancer effects even by intravenous administration, thereby leading to completion of the present invention.

In addition, the inventors of the present invention also found that, even if isopropyl alcohol, which is an inhibitor of self-digestion by bacillolysin MA, is not used during production of BL-angiostatin, BL-angiostatin can still be produced efficiently, thereby leading to completion of the present invention.

### DISCLOSURE OF THF INVENTION

The present invention relates to an anticancer agent containing as its active ingredient BL-angiostatin, and a production method thereof.

### BRIEF DESCRIPTION OF THE DRAWINGS

FIG. 1 shows BL-angiostatin purified using an affinity trap reactor. Lane 1 indicates the result of analyzing the resulting BL-angiostatin. Lane 2 indicates the result of incubating bacillolysin MA (5 nM) and plasminogen (3 μM) for 60 minutes at 37° C. in 10 μl of 50 mM Tris-HCl (pH 7.4) containing 100 mM NaCl, 0.01% Tween80 and 1 mM CaCl₂, followed by adding 10 μl of sample buffer (125 mM Tris-HCl buffer (pH 6.8) containing 4% SDS, 10% 2-mercaptoethanol, 20% sucrose and 0.004% bromphenol blue) and analyzing in the same manner as Lane 1.

FIG. 2 is a graph indicating the effects of BL-angiostatin on the proliferation on subcutaneously transplanted Lewis lung carcinoma (changes in tumor volume). The number of days (days) from transplant of Lewis lung carcinoma is indicated on the X axis, while tumor volume (mm³) is indicated on the Y axis. The indicates a control group, while the ▴, ▪, X and ♦ indicate BL-angiostatin treatment groups dosed at 0.3, 1, 3 and 10 mg/kg/day, respectively. Each value represents the mean±standard deviation of 8 animals in each group. Statistically significant differences as compared with the control group based on Dunnett's multiple comparison test are indicated with asterisks (*=p<0.05, **=p<0.01).

FIG. 3 is a graph indicating the effects of BL-angiostatin on the proliferation of subcutaneously transplanted Lewis lung carcinoma (tumor wet weight). The dosage (mg/kg/day) of BL-angiostatin is indicated on the X axis, while the tumor wet weight (mg) is indicated on the Y axis. Each value represents the mean±standard deviation of 8 animals in each group. Statistically significant differences as compared with the control group based on Dunnett's multiple comparison test are indicated with asterisks (*=p<0.05, **=p<0.01).

FIG. 4 is a graph indicating the effects of BL-angiostatin on the body weights of animals subcutaneously transplanted with Lewis lung carcinoma. The number of days (days) from transplant of Lewis lung carcinoma is indicated on the X axis, while body weights (g) are indicated on the Y axis. The indicates a control group (physiological saline), ▴ indicates an angiostatin 0.3 mg/kg/day treatment group, ▪ indicates an angiostatin 1 mg/kg/day treatment group, X indicates an angiostatin 3 mg/kg/day treatment group, and ♦ indicates an angiostatin 10 mg/kg/day treatment group. Each value represents the mean±standard deviation of 8 animals in each group. Statistically significant differences as compared with the control group based on Dunnett's multiple comparison test are indicated with asterisks (*=p<0.05, **=p<0.01).

FIG. 5 shows tumor histopathological images and images resulting from von Willebrand factor immunostaining. Representative examples are shown for a control group (tumor major axis length: 8.2 mm, minor axis length: 7.8 mm) (A, B, C and D), a 3 mg/kg dose group (tumor major axis length: 7.0 mm, minor axis length: 5.6 mm) (E, F, G and H), and a 10 mg/kg dose group (tumor major axis length: 4.4 mm, minor axis length: 2.8 mm) (I, J, K and L). D, H and L indicate the results of von Willebrand factor staining, while the others shows the results of hematoxylin-eosin staining. The magnification factors are 5× (A, E, I), 25× (B, F, J), 100× (C, G, K) and 50× (D, H, L).

### BEST MODE FOR CARRYING OUT THF INVENTION

### BL-Angiostatin of the Present Invention

The BL-angiostatin of the present invention can be obtained by treating plasminogen using a protease, and particularly bacillolysin MA (BLMA), which is an enzyme produced by Bacillus megaterium strain A9542. Bacillus megaterium strain A9542 was deposited at the International Patented Organism Depository of the National Institute of Bioscience and Human Technology, National Institute of Advanced Industrial Science and Technology on Mar. 21, 2001 under accession number FERM P-18268. The amino acid sequence of BLMA is disclosed in Japanese Unexamined Patent Publication No. 2002-272453.

BLMA is known to produce four types of fragments having angiostatin-like activity consisting of Glu¹-Ser⁴⁴¹, Glu¹-Val⁴⁴⁹, Phe⁷⁵-Ser⁴⁴¹ and Phe⁷⁵-Val⁴⁴⁹ as a result of cleaving between Ser⁴⁴¹-Val⁴⁴², Leu⁷⁴-Phe⁷⁵ and Val⁴⁴⁹-Leu⁴⁵⁰ of human plasminogen.

Although one or more of the aforementioned four types of angiostatin-like fragments can be used alone or in combination for the anticancer agent of the present invention, Glu¹-Ser⁴⁴¹ is used particularly preferably.

The amino acid sequence of plasminogen is known to be preserved to a high degree in mammals, and particularly in humans and bovines. Thus, all types of plasminogen originating in mammals can be used as a raw material for producing the BL-angiostatin of the present invention, while plasminogen of human or bovine origin is particularly preferable.

In addition, the BL-angiostatin of the present invention can also be produced by chemical synthesis or gene recombination technology. Production using gene recombination technology is preferable in terms of the procedure being comparatively easy and enabling BL-angiostatin to be produced in large quantities. When producing the BL-angiostatin of the present invention, DNA having a nucleotide sequence which encodes said protein is produced followed by producing the target protein by inserting said DNA into a suitable expression system.

DNA having the nucleotide sequence of the BL-angiostatin of the present invention can be acquired by screening a cDNA library of human origin (e.g., derived from HepG2 cells) using a primer or probe suitably designed based on information of a nucleotide sequence which encodes the amino acid sequence of BL-angiostatin. Screening can be carried out by plaque hybridization and so forth. Alternatively, the target gene can be cloned directly by carrying out PCR using a cDNA library or human origin (e.g., derived from HepG2 cells) as a template and using a primer suitably designed based on information on the aforementioned nucleotide sequence.

Expression systems for expressing a recombinant protein (consisting of an expression vector containing a gene and a host) are known among persons with ordinary skill in the art. In order to express DNA in host cells, said DNA is first inserted downstream from a promoter in an expression vector, followed by inserting the recombinant expression vector into host cells compatible with said expression vector.

Examples of bacteria expression vectors include pGEMEX-1 (Promega), pQE-9 (Qiagen), pQE-30 (Qiagen), pRSET (Invitrogen) and pLEX (Invitrogen), examples of animal cell expression vectors include pcDNAI, pcDM8 (Funakoshi), pcDNAI/AmP (Invitrogen) and pREP4 (Invitrogen), while examples of expression vectors for recombinant virus production include pMFG (Takara).

Examples of promoters which can be used for bacteria expression vectors include promoters originating in Escherichia coli or phages such as trp promoter, T7 promoter and lac promoter. Examples of promoters which can be used for yeast expression vectors include PHO5 promoter and GAP promoter. Examples of promoter that can be used for animal cell expression vectors include cytomegalovirus (CMV) IE (immediate early) gene promoter, SV40 early promoter, retrovirus promoter, adenovirus promoter, metallotionein promoter and heat shock promoter.

There are no particular limitations on the host cells provided they are able to express the target protein, and examples include bacteria, yeast, animals cells and insect cells.

Examples of methods for inserting a recombinant vector into a host include the calcium phosphate method, protoplast method, electroporation, spheroblast method, lithium acetate method and lipofection, and the method can be suitably selected corresponding to the type of host cells.

Ordinary protein isolation and purification methods can be used to isolate and purify the target recombinant protein from a transformant culture. For example, in the case the recombinant protein is expressed in the form of a solution in cells, following completion of culturing, the cells are collected by centrifugation and then suspended into an aqueous buffer, followed by destruction of the cells to obtain a cell-free extract. Following centrifugal separation of the cell-free extract, the target recombinant protein can be purified from the resulting supernatant using ordinary protein isolation and purification methods, namely solvent extraction, salting-out with ammonium sulfate, desalination, precipitation with organic solvent, ion exchange chromatography, gel filtration or affinity chromatography, either alone or in a combination thereof.

In addition, the present invention relates to a method for producing BL-angiostatin comprising the steps of:

equilibrating an affinity trap reactor, in which bacillolysin MA and lysine are immobilized on agarose gel, with a buffer;

adding citric acid-treated serum;

washing with a buffer containing sodium chloride; and

eluting with 6-aminohexanoic acid; wherein,

the buffer does not contain isopropyl alcohol.

Preferably, all of the procedures are carried out at 4° C. As a result, BL-angiostatin can be obtained at high yield.

The anticancer agent of the present invention is typically provided in the form of a pharmaceutical composition containing BL-angiostatin as active ingredient and a pharmaceutical additive such as a excipient or a vehicle.

The anticancer agent of the present invention can be administered as a pharmaceutical to mammals, including humans.

There are no particular limitations on the administration route of the pharmaceutical preparation of the present invention, it can be administered by oral administration or parenteral administration (such as intramuscular administration, intravenous administration, subcutaneous administration, intraperitoneal administration, mucosal membrane administration such as into the nasal cavity, or inhalation administration).

Differing from the angiostatin of the prior art, since BL-angiostatin has high solubility in water (20 mg/ml or more), the anticancer agent of the present invention is characterized by being able to be administered by intravenous administration.

There are no particular limitations on the form of the anticancer agent of the present invention, and examples of preparations for oral administration include tablets, capsules, grains, powders, granules, liquids and syrups, while examples of preparations for parenteral administration include injections, infusions, suppositories, inhalants, transmucosal absorbents, transcutaneous absorbents, nose drops and ear drops. The drug form of the present invention, preparation additives to be used, preparation production method and so forth can be suitably selected by a person with ordinary skill in the art.

The dosage of the pharmaceutical of the present invention can be suitably selected by comprehensively taking into consideration the gender, age or body weight of the patient, severity of symptoms, purpose of administration such as for prevention or treatment, or the presence of other complications and so forth. The dosage is typically 0.1 mg/kg of body weight/day to 100 mg/kg of body weight/day, and preferably 1 mg/kg of body weight/day to 10 mg/kg of body weight/day.

The anticancer agent of the present invention can be used for the treatment of cancers such as breast cancer, lung cancer, pharynx cancer, stomach cancer, pancreas cancer, liver cancer, colon cancer, uterine cancer and ovarian cancer.

Although the following provides a detailed explanation of the present invention, the present invention is not limited to that described in the examples.

### Example 1

### Production and Purification of Bacillolysin MA

Bacillolysin MA was isolated and purified according to the method described below from Bacillus megaterium strain A9542 (deposited at the International Patented Organism Depository of the National Institute of Bioscience and Human Technology, National Institute of Advanced Industrial Science and Technology under accession number FERM P-18268).

After culturing Bacillus megaterium strain A9542 by shake culturing for 6 days at 28° C. in a 500 ml volumetric Erlenmeyer flask containing 100 ml of a liquid medium (pH 7.0) comprised of 1% glucose, 3% cornstarch, 1% soybean meal, 0.5% peptone, 0.5% yeast extract, 0.2% CaCO₃ and 0.01% CB442, 3 liters of the culture liquid were filtered using celite, and 1 liter of the resulting filtrate was diluted with 5 liters of water followed by the addition of isopropyl alcohol to a final concentration of 5% (v/v). Subsequently, the product was injected at a flow rate of 15 ml/min into a 400 ml carboxymethyl cellulose (Seikagaku) column equilibrated with 20 mM MES (2-[N-morpholino]ethanesulfonic acid)-NaOH buffer (pH 6.5)/5% isopropyl alcohol. After washing the column with 600 ml of the same buffer, the column was eluted with 20 mM MES/NaOH (pH 6.5)/5% isopropyl alcohol/0.2 M NaCl. The eluted fractions were fractioned in 60 ml aliquots, and those fractions that demonstrated activity were collected. Purity was confirmed by SDS-PAGE and 90 mg of purified product were obtained.

### Example 2

### Production of Affinity Trap Reactor of Present Invention Immobilized with Bacillolysin MA and Lysine (Production of 10 ml of Reactor)

2.86 g of agarose gel (Sepharose 4B, Pharmacia) pre-activated with cyanogen bromide were suspended in 100 ml of 1 mM HCl solution followed by stirring for 15 minutes at room temperature. This suspension was then transferred to a column and the HCl solution was removed by aspiration followed by washing the gel three times with 100 ml of 1 mM HCl solution and once with 75 ml of buffer A (0.1 M sodium bicarbonate, pH 8.3) containing 0.5 M NaCl and 5% isopropyl alcohol. A 2.84 mg/ml bacillolysin MA solution was prepared with the same buffer A and 17.6 ml of this solution were added to a column to carry out the immobilization reaction by stirring for 2 hours at room temperature. Following completion of the reaction, the solution was removed by aspiration followed by the addition of 20 ml of 5% aqueous isopropyl alcohol solution containing 0.2 M L-lysine hydrochloride (pH 8.0) to carry out the lysine immobilization reaction by stirring for 2 hours at room temperature. Following completion of the reaction, the solvent was removed by aspiration followed by washing the gel with 500 ml of buffer B (20 mM MES (2-[morpholino]ethane sulfonic acid)-NaOH) containing 5% isopropyl alcohol, and finally storing at 4° C. in buffer B having the aforementioned composition containing 0.02% sodium azide.

### Example 3

### Single-Stage Purification Process of BL-Angiostatin from Human Plasma Using a Bacillolysin MA/Lysine Reactor

After adding 5 ml of isopropyl alcohol to 95 ml of human plasma treated with citric acid on ice and allowing to stand for 30 minutes, the supernatant was obtained by centrifugal separation (22,000×g, 4° C., 1 hour) and then filtered. The remainder of the procedure was carried out at 4° C. 10 ml of the bacillolysin MA/lysine reactor of the present invention produced in Example 2 were equilibrated with buffer C (25 mM sodium phosphate, pH 7.4) containing 5% isopropyl alcohol. The aforementioned supernatant was then added to the equilibrated reactor at a flow rate of 1.5 ml/min. Subsequently, the reactor was washed with 200 ml of buffer C having the aforementioned composition containing 0.5 M NaCl (3 ml/min). Elution of the resulting BL-angiostatin was carried out with 50 ml of 5% isopropyl alcohol solution containing 200 mM 6-aminohexanoic acid. As a result, nearly all of the BL-angiostatin eluted in 10 to 30 ml of eluate. The yield of the BL-angiostatin was 4.2 mg, and the yield was 95%.

Furthermore, after the reactor was used, it was washed at 4° C. using 50 ml of buffer C having the aforementioned composition containing 1 M NaCl and 200 mM 6-aminohexanoic acid. Subsequently, it was stored at 4° C. in buffer C containing 0.02% sodium azide. When washed and stored in this state, the reactor was able to be used repeatedly for 2 months or more.

### Example 4

### Single-Stage Purification Process of BL-Angiostatin from Human Plasma Using Bacillolysin MA/Lysine Reactor (Method Not Using Isopropyl Alcohol)

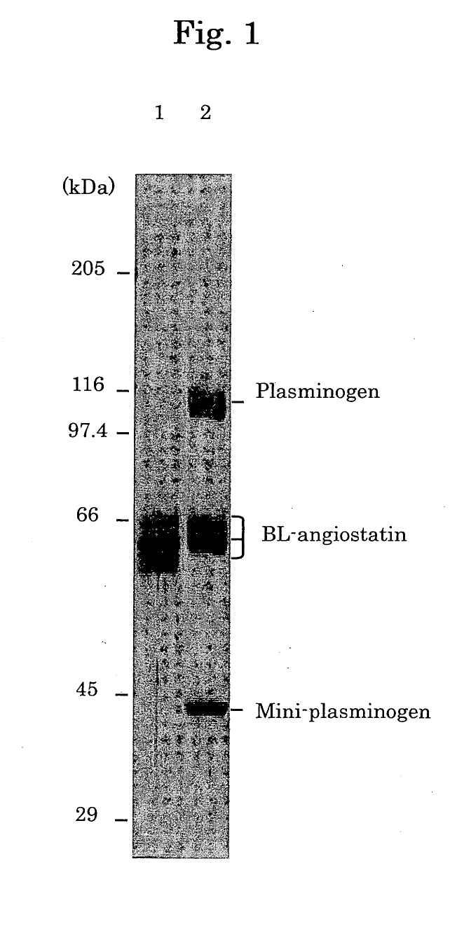

Human plasma treated with citric acid was centrifuged for 20 minutes at 15,000 rpm to obtain supernatant. The remainder of the procedure was entirely carried out at 4° C. 0.5 ml of the bacillolysin MA/lysine reactor of the present invention produced in Example 2 were equilibrated with buffer C. 2.5 ml of the aforementioned supernatant were then added thereto at a flow rate of 0.1 ml/min. Subsequently, after washing with 15 ml of buffer C containing 0.5 M sodium chloride (0.15 ml/min), the reactor was eluted with 2.5 ml of 200 mM 6-aminohexanoic acid. The majority of the BL-angiostatin was eluted in 0 to 1.5 ml of eluate. The yield of BL-angiostatin was 63 μg, and the purity was 94% (FIG. 1).

Furthermore, after the reactor was used, it was washed using 2.5 ml of buffer C containing 1 M NaCl and 200 mM 6-aminohexanoic acid. Subsequently, it was stored at 4° C. in buffer C containing 0.02% sodium azide. When washed and stored in this state, the reactor was able to be used repeatedly for 2 months or more.

4 μg of the resulting BL-angiostatin was dissolved in purified water and brought to a final volume of 10 μl. 10 μl of sample buffer (125 mM Tris-HCl buffer containing 4% SDS, 10% 2-mercaptoethanol, 20% sucrose and 0.004% bromphenol blue, pH 6.8) were then added thereto, of which 10 μl were fractionated by electrophoresis using 7.5% polyacrylamide gel followed by staining with Coomassie brilliant blue R250 (FIG. 1).

### Example 5

### Effects of BL-Angiostatin on Lewis Lung Carcinoma

* Cancer Type, Cancer Transplant Method and Experimental Animals

Lewis lung carcinoma cells were sub-cultured by subcutaneously transplanted into C57BL/6 mice. The tumors were excised on days 10 to 14 and the carcinoma cells were suspended in phosphate-buffered physiological saline. 0.05 ml of the cell suspension containing 1×10⁵ cells per animal were transplanted beneath the skin in the ventral region of BDF₁ mice purchased at age 5 weeks and subsequently preliminarily housed for 1 week in an SPF environment. The remainder of the experiment was carried out in an SPF environment.

* Pharmaceutical Preparation Method, Administration Method and Administration Conditions

BL-angiostatin prepared according to the method described in Example 3 was freeze-dried and then dialyzed against purified water three times for 2 hours. The resulting solution was then freeze-dried and dissolved in physiological saline to a concentration of 20 mg/ml. This solution was sterilized by filtration using a filter having a pore diameter of 0.22 μl. This solution was additionally diluted with sterile physiological saline to prepare solutions having concentrations of 0.06, 0.2, 0.6 and 2 mg/ml. The aforementioned BL-angiostatin solutions were administered intravenously for 14 days once a day at the rate of 5 ml/kg starting on the day after transplantation of the Lewis lung carcinoma cells. As a result, the resulting dosages were 0.3, 1, 3 and 10 mg/kg. A control group was intravenously administered an equal volume of physiological saline. Ten animals were assigned to each group.

* Observed Parameters

Tumor size (tumor volume was calculated in the following manner from the minor and major axes: (minor axis)²×major axis×0.52) and body weight were measured daily during the dosing period. On the day following completion of dosing, the mice were sacrificed under ether anesthesia and the tumors were excised followed by measurement of their wet weight. After omitting the maximum and minimum values of each group, the data of a total of 8 animals was subjected to statistical processing. Dunnett's multiple comparison test was used to test for the presence of a significant difference, and a level of significance of 0.05% or less was considered to be significant. Moreover, measurement of the major and minor axes, observation of cell proliferation, bleeding and necrosis, histological observation of dividing cells and immunostaining of vascular endothelial cells using anti-von Willebrand factor antibody were carried out on the tumors obtained from three representative animals each of the control group, 3 mg/kg dose group and 10 mg/kg dose group.

* Observation Results and Discussion

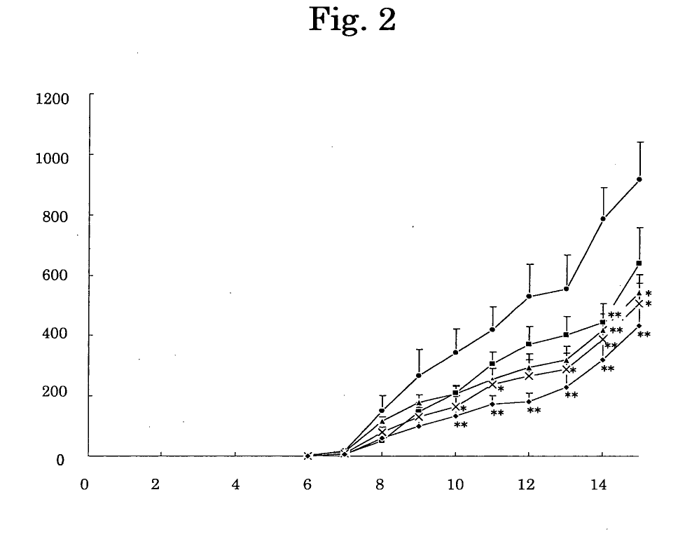

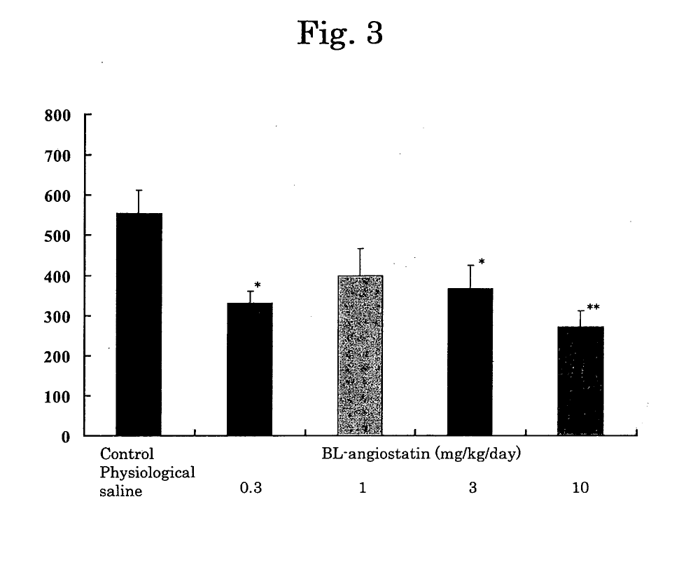

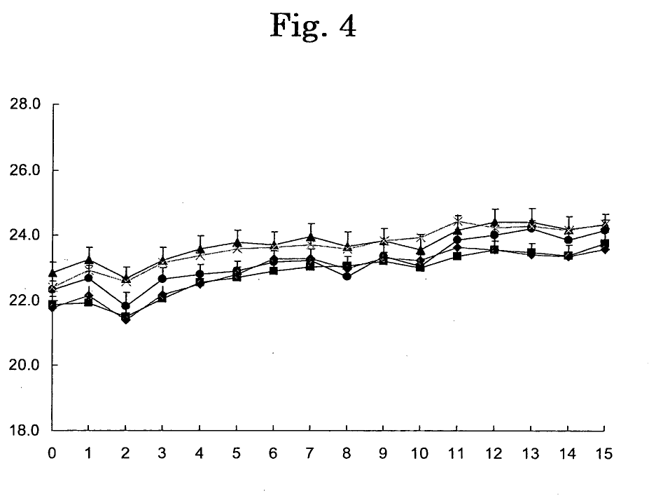

Tumor volume decreased significantly in the 3 and 10 mg/kg BL-angiostatin dose groups on day 10 following transplant of Lewis lung carcinoma as compared with the control group (FIG. 2). In the 10 mg/kg dose group, tumor volume was ½ or less that of the control group throughout the observation period. Although inhibition of tumor growth was observed in the 0.3 and 1 mg/kg dose groups as well, statistically significant differences were observed on days 14 and 15 in the 0.3 mg/kg dose group and on day 14 in the 1 mg/kg dose group (FIG. 2). In addition, although the tumors were excised on day 15 following transplant and their wet weights were measured, the values in the BL-angiostatin dose groups were 50 to 70% those of the control group (FIG. 3). There were no statistically significant differences observed among the 0.3, 3 and 10 mg/kg dose groups. On the other hand, there were no significant differences observed in the body weights of each group throughout the period the animals were housed (FIG. 4). In addition, there were also no differences observed in the general conditions of the animals among the study groups.

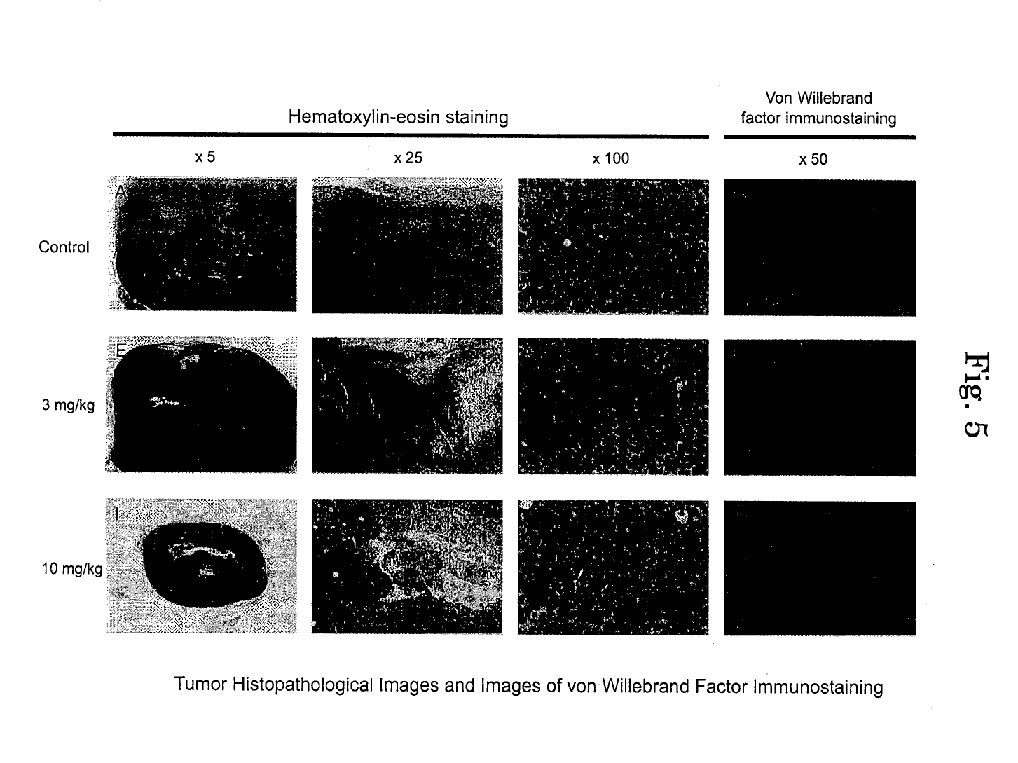

The results of histopathological examinations performed on the excised tumors are summarized in Table 1. Advanced cell proliferation (FIG. 5A), mild bleeding (FIG. 5B), slight necrosis (FIG. 5B) and moderate dividing cells (FIG. 5C) were observed in the control group. In addition, immunostaining of vascular endothelial cells with anti-von Willebrand factor antibody revealed a mild to moderate proportion of positive cells (FIG. 5D). Moderate cell proliferation (FIG. 5E), mild bleeding (FIG. 5F), mild necrosis (FIG. 5F), moderate dividing cells (FIG. 5G) and a mild to moderate proportion of von Willebrand factor positive cells (FIG. 5H) were observed in the BL-angiostatin 3 mg/kg dose group. Moreover, mild to moderate cell proliferation (FIG. 5I), mild to moderate bleeding (FIG. 5J), mild to severe necrosis (FIG. 5J), slight to mild dividing cells (FIG. 5K) and a slight to mild proportion of von Willebrand factor positive cells (FIG. 5L) were observed in the BL-angiostatin 10 mg/kg dose group.

TABLE 1
Results of Tumor Histopathological Examinations and
Von Willebrand Factor Immunostaining

| Dosage (mg/kg) | 0 | | | 3 | | | 10 | | |
|---|---|---|---|---|---|---|---|---|---|
| Tumor histopatho- | | | | | | | | | |
| logical findings | | | | | | | | | |
| (hematoxylin-eosin | | | | | | | | | |
| staining) | | | | | | | | | |
| Major axis | 10.6 | 7.6 | 8.2 | 7.4 | 7.0 | 7.1 | 5.5 | 6.7 | 4.4 |
| Minor axis | 5.0 | 7.5 | 7.8 | 5.6 | 5.6 | 4.2 | 2.8 | 6.2 | 2.8 |
| Cell proliferation | +++ | +++ | +++ | ++ | ++ | ++ | ± | ++ | + |
| Bleeding | + | + | + | + | + | + | ++ | ± | + |
| Necrosis | ± | − | ± | + | + | + | +++ | + | ++ |
| Dividing cells | ++ | ++ | ++ | ++ | ++ | ++ | ± | + | ± |
| Tumor von | + | ++ | ++ | + | ++ | ± | ± | + | ± |
| Willebrand factor | | | | | | | | | |
| immunostaining | | | | | | | | | |
| findings (proportion | | | | | | | | | |
| of positive vessels) | | | | | | | | | |

On the basis of these results, intravenous administration of BL-angiostatin at 0.3 to 10 mg/kg/day was indicated as being effective in inhibiting the growth of subcutaneously transplanted Lewis lung carcinoma. In addition, the results of tumor histopathological examination of the tumors indicated administration of BL-angiostatin decreases division and proliferation of tumor cells while also causing increased necrosis. Moreover, as a result of immunostaining vascular endothelial cells with anti-von Willebrand factor antibody, BL-angiostatin was confirmed to inhibit cancer-associated vascularization, and this vascularization inhibitory action is thought to bring about the aforementioned decreases in division and proliferation of tumor cells and the increased necrosis. In addition, since there were no effects on the body weights or general conditions of the experimental animals, BL-angiostatin is presumed to not cause any serious adverse side effects. Since the growth of Lewis lung carcinoma, which has superior clinical predictability, was inhibited, inhibitory effects are considered to be demonstrated against numerous other cancers as well (including breast cancer, lung cancer, pharynx cancer, pancreas cancer, liver cancer, colon cancer, uterine cancer and ovarian cancer).

### INDUSTRIAL APPLICABILITY

The anticancer agent of the present invention can be used to treat cancers such as breast cancer, lung cancer, pharynx cancer, stomach cancer, pancreas cancer, liver cancer, colon cancer, uterine cancer, ovarian cancer and prostate cancer.

1. An anticancer composition comprising a pharmaceutically effective amount of BL-angiostatin as an active ingredient thereof in combination with a pharmaceutically acceptable carrier.

2. The anticancer composition according claim 1 wherein the BL-angiostatin is derived from a mammal.

3. The anticancer composition according to claim 1 wherein the BL-angiostatin comprises one or more fragments of human plasminogen selected from the group consisting of Glu¹-Ser⁴⁴¹, Glu¹-Val⁴⁴⁹, Phe⁷⁵-Ser⁴⁴¹ and Phe⁷⁵-Val⁴⁴⁹.

4. A method for producing the BL-angiostatin according to claim 1 comprising the steps of:
equilibrating an affinity trap reactor, in which bacillolysin MA and lysine are immobilized on agarose gel, with a buffer;
adding serum treated with citric acid;
washing with a buffer containing sodium chloride; and
eluting with 6-aminohexanoic acid; wherein,
the buffer does not contain isopropyl alcohol.

5. The anticancer composition according to claim 2, wherein the BL-angiostatin comprises one or more fragments of human plasminogen selected from the group consisting of Glu¹-Ser⁴⁴¹, Glu¹-Val⁴⁴⁹, Phe⁷⁵-Ser⁴⁴¹ and Phe⁷⁵-Val⁴⁴⁹.

6. A method for treating a cancer comprising administering to a mammal a pharmaceutically effective amount of BL-angiostatin.

7. The method for treating cancer according to claim 6, wherein the mammal is a human.

8. The method for treating cancer according to claim 7, wherein the cancer is breast cancer.

9. The method for treating cancer according to claim 7, wherein the cancer is lung cancer.

10. The method for treating cancer according to claim 7, wherein the cancer is pharynx cancer.

11. The method for treating cancer according to claim 7, wherein the cancer is stomach cancer.

12. The method for treating cancer according to claim 7, wherein the cancer is pancreas cancer.

13. The method for treating cancer according to claim 7, wherein the cancer is liver cancer.

14. The method for treating cancer according to claim 7, wherein the cancer is colon cancer.

15. The method for treating cancer according to claim 7, wherein the cancer is uterine cancer.

16. The method for treating cancer according to claim 7, wherein the cancer is ovarian cancer.

17. The method for treating cancer according to claim 7, wherein the BL-angiostatin is administered in a dosage of 0.1 mg/kg of body weight/day to 100 mg/kg of body weight/day.

18. The method for treating cancer according to claim 17, wherein the dosage is 1 mg/kg of body weight/day to 10 mg/kg of body weight/day.

19. The method for treating cancer according to claim 7, wherein the BL-angiostatin comprises a fragment of human plasminogen which is Glu¹-Ser⁴⁴¹.
